(19) United States
(12) Patent Application Publication
(10) Pub. No.: US 2009/0246787 A1
(43) Pub. Date: Oct. 1, 2009

(54) METHOD AND APPARATUS FOR DETERMINING CHEMOSENSITIVITY

(75) Inventors: Tomoko Oyama, Akashi-shi (JP); Tomokazu Yoshida, Kobe-shi (JP); Satoshi Nakayama, Kobe-shi (JP); Yumi Takinowaki, Suntou-gun (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/407,301

(22) Filed: Mar. 19, 2009

(30) Foreign Application Priority Data
Mar. 31, 2008 (JP) 2008-091443

(51) Int. Cl. C12Q 1/68; C12Q 1/02; C12M 1/00

(52) U.S. Cl. 435/6; 435/29; 435/287.2

(57) ABSTRACT

A method for determining chemosensitivity of a malignant tumor cell to anthracycline is described herein. According to this method, first tumor cell and second tumor sell are first prepared. The first tumor cell is not treated with anthracycline, and on the other the second tumor cell is treated with anthracycline. Secondly, first and second activity levels of CDK1 are measured. The first activity level is activity of CDK1 in the first tumor cell and the second activity level is that of CDK1 in the second tumor cell. Finally, chemosensitivity of the malignant tumor cell is determined. The determination is based on the first and second activity levels.

### TECHNICAL FIELD

The present invention relates to method and apparatus for determining chemosensitivity of malignant tumor cell to anticancer agent “anthracycline.”

### BACKGROUND

As a known method of judging the effectiveness of chemotherapy or the chemosensitivity of cells to an anticancer agent, there is a method of using, as a judgment parameter, at least one factor selected from the expression level of a cyclin-dependent kinase (hereinafter referred to as CDK), the activity level thereof, and the ratio of the expression level to the activity level thereof.

For example, U.S. Patent Application Publication No. 2007-077658 discloses a method of judging the chemosensitivity to a taxane-based anticancer agent, which comprises measuring the specific activity of CDK2 and the expression level of p21 in a cancer tissue extirpated from a patient and comparing these parameters with a predetermined threshold value. In addition, a method of judging the chemosensitivity of CE therapy (combination therapy with cyclophosphamide and epirubicin) by comparing the ratio between the specific activity of CDK2 and the expression level of cyclin E with a predetermined threshold value is also disclosed.

### SUMMARY

The object of the present invention is to provide a method and an apparatus capable of determining the chemosensitivity of a malignant tumor cell to anthracycline.

The method for determining chemosensitivity of malignant tumor cell to anthracycline comprises steps of preparing first and second tumor cells, measuring first activity level, measuring second activity level, and determining chemosensitivity of the malignant tumor cell. In this method, the first tumor cell and second tumor cell are obtained from a subject. The first tumor cell is not treated with anthracycline, on the other the second tumor cell is treated. The first activity level is activity level of CDK 1 present in the first tumor cell, and the second activity level is activity level of CDK1 present in the second tumor cell. The determination of chemosensitive is based on the first and second activity level.

The apparatus for determining chemosensitivity of malignant tumor cell to anthracycline comprises an information obtaining part, a determining part and a display. The information obtaining part obtains first and second information. The first information is related to activity level of CDK1 present in the first tumor cell, and the second information is related to activity level of CDK1 present in the second tumor cell. The determining part determines chemosensitivity of the malignant tumor cell based on the first and second information. The display outputs result of the determining.

### BRIEF DESCRIPTION OF THE DRAWINGS

FIG. 1 shows the results of Reference Example 2.

FIG. 2 shows the results of Example 1.

FIG. 3 shows the results of Example 2.

FIG. 4 shows the results of Example 3.

FIG. 5 shows the results of Example 4.

FIG. 6 shows the results of Comparative Example 1.

FIG. 7 shows the results of Comparative Example 2.

FIG. 8 shows the results of Comparative Example 3.

FIG. 9 shows the results of Comparative Example 4.

FIG. 10 is a block diagram showing a schematic configuration of the apparatus of the embodiment.

FIG. 11 is a flowchart showing processing executed by the apparatus shown in FIG. 10.

FIG. 12 shows a screen (window) displayed in the display unit 105 in the apparatus 1 shown in FIG. 10.

### DESCRIPTION OF THE PREFERRED EMBODIMENTS

### 1. Method of the Invention

### Embodiment 1

The method for determining chemosensitivity of a malignant tumor cell to anthracycline in one embodiment of the invention (Embodiment 1) comprises preparing . . . . (Claim)

First, in the preparing step of the method in accordance with Embodiment 1, a first tumor cell and a second tumor cell are prepared. Both the first and second tumor cells are malignant tumor cells derived from a malignant tumor in a subject.

The malignant tumor cells include, for example, breast cancer cells, lung cancer cells, hepatic cancer cells, stomach cancer cells, colon cancer cells, uterine cervix cancer cells, ovarian cancer cells, pancreas cancer cells, prostate cancer cells, skin cancer cells, brain tumor cells, leukemic cells and the like. These malignant tumor cells derived from tumor tissues of malignant tumor patients (subjects). Among these malignant tumor cells, breast cancer cells are preferable in the method of the present embodiment.

The first tumor cell is prepared without treatment with an anticancer agent. For example, the first tumor cell can be obtained by culturing a malignant tumor cell in a container including an anticancer agent-free medium.

The medium may be any medium suitable for growth of tumor cells. Examples of the medium include mediums containing, as a basal medium, a Dulbecco's modified medium, an Eagle minimum essential medium, an RPMI 1640 medium, a Liebovitz L15 medium, or the like. These mediums maybe those supplemented if desired with complementary additives such as L-glutamine, fetal bovine serum, insulin, glucose and sodium bicarbonate. The concentrations of these complementary additives in the medium can be appropriately established depending on the type of tissue from which the tumor cells have been collected, the type of cancer, and the type of the complementary additive.

The conditions for culturing tumor cells in the medium are not sweepingly generalized and vary depending on the type of tissue from which the tumor cells have been collected, the type of malignant tumor, and the like. For example, the tumor cells can be cultured in 5 vol % carbon dioxide at 37° C.

When tumor cells have been cultured in a medium, the tumor cells in a container are washed, and the washed tumor cells are detached from the container. For example, phosphate buffered physiological saline, or physiological saline containing a buffer such as Tris or HEPES, can be used to wash the tumor cells. The tumor cells can be detached from the container, for example by adding a cell dispersing reagent such as a trypsin-EDTA solution (composition: 0.25% by mass of trypsin and 1 mM of EDTA) to the washed tumor cells and then incubating the cells. The detached tumor cells may be recovered after washing with, for example, phosphate buffered physiological saline.

In the first measuring step, the activity level of CDK1 contained in the recovered tumor cell is measured. The CDK1 activity level obtained from this first tumor cell is referred to as first activity level.

The activity level of CDK1 is measured in terms of kinase activity level (unit) calculated from the amount of a substrate phosphorylated by binding to a specific cyclin. The substrate to be phosphorylated with CDK1 includes, for example, histone H1.

The activity level of CDK1 can be measured by a conventional method of measuring CDK activity. For example, there is a method of using a radioactive label or a method of using no radioactive label. Specifically, the method of using a radioactive label includes a method which comprises preparing a sample containing activated CDK1 from a cell lysate, then using the sample and ³²P-labeled ATP (γ-[³²P]-ATP) so that a substrate protein is allowed to incorporate ³²P, measuring the labeling amount of the ³²P-labeled phosphorylated substrate, and quantitatively determining the activity level based on a standard curve previously prepared using standard samples. The method using no radioactive label includes a method described in U.S. Patent Application Publication No. 2002-164673. The method described in U.S. Patent Application Publication No. 2002-164673 is a method which comprises preparing a sample containing activated CDK1 from a cell lysate as a sample to be measured, reacting the substrate protein in the sample with adenosine 5′-O-(3-thiotriphosphate) (ATP-γS) to introduce a monothiophosphate group into a serine or threonine residue of the substrate protein, binding a fluorescent substance or a labeled enzyme to a sulfur atom in the introduced monothiophosphate group thereby labeling the substrate protein, measuring the amount of the labeled thiophosphorylated substrate (or the amount of the fluorescent substance in the case where the fluorescent substance is used), and quantitatively determining the activity level based on a standard curve previously prepared using standard samples.

The sample to be measured for the activity level of CDK1 is prepared by collecting CDK1 specifically from a cell lysate of tumor cells to be measured. The cell lysate of tumor cells can be obtained by solubilizing the tumor cells with a surfactant-containing solution or the like. The sample to be measured for the activity level of CDK1 can be prepared by immunoprecipitation with an anti-CDK1 antibody, an anti-CDK1/cyclin A conjugated antibody, an anti-CDK1/cyclin B conjugated antibody, and the like. The sample may contain CDK other than activated CDK1. The sample also contains e.g. conjugates having a CDK inhibitor bound to a cyclin A/CDK1 conjugate or a cyclin B/CDK1 conjugate. The sample further contains CDK1 itself, CDK1/cyclin (cyclin A or B) conjugates, CDK1/cyclin/CDK inhibitor conjugates, and conjugates of CDK1 and other compounds. Accordingly, the activity level is measured in terms of the unit (U) of the phosphorylated substrate under the condition where various CDKs such as activated CDK1, inactivated CDK1, and various competitive reactive substances coexist.

The second tumor cell is prepared by treating a tumor cell with anthracycline. For example, the second tumor cell can be obtained by culturing a tumor cell in a container including an anthracycline-containing medium. The concentration of anthracycline in the medium is preferably 0.1 nM to 10000 nM, and more preferably 10 nM to 1000 nM.

Anthracycline is an anthracycline-based compound having an anticancer action. Preferable examples include daunorubicin, doxorubicin, pirarubicin, aclarubicin, epirubicin, oxaunomycin, and idarubicin.

After treatment of the tumor cell with anthracycline, the tumor cell is recovered by the same operation as described above in measurement of the activity level. In the second measuring step, the activity level of CDK1 contained in the recovered tumor cell is measured. The CDK1 activity level obtained from the second tumor cell is referred to as second activity level.

In the determining step thereafter, the chemosensitivity of the tumor cell to anthracycline is judged based on the first and second activity levels. Preferably, the chemosensitivity is judged based on the degree of change between the first activity level and the second activity level. The tumor cell that is chemosensitive to anthracycline and the tumor cell that is not chemosensitive to anthracycline are significantly different in this degree of change.

When the tumor cell chemosensitive to anthracycline is treated with anthracycline, the activity level of CDK1 in the cell is significantly decreased. On the other hand, the tumor cell not chemosensitive to anthracycline, even if treated with anthracycline, does not show a significant decrease in the activity level of CDK1. According to this embodiment, it can be judged that when the activity of CDK1 in the tumor cell is decreased more than a certain level or more than a certain ratio by treatment with anthracycline, the tumor cell is chemosensitive to anthracycline. When the decrease in the activity of CDK1 by treatment with anthracycline is less than a certain level or less than a certain ratio, when the activity level of CDK1 is not changed even by treatment with anthracycline, or when the activity level of CDK1 increases, the tumor cell can be judged to be not chemosensitive to anthracycline.

In this specification, the degree of change in activity level indicates the degree of change from first activity level to second activity level (or the degree of change from second activity level to first activity level). The degree of change in activity level is for example the difference between first activity level and second activity level, the ratio between second activity level and first activity level, or the like. The difference between first activity level and second activity level includes ([first activity level]−[second activity level]) and ([second activity level]−[first activity level]). The ratio between first activity level and second activity level includes the ratio of second activity level to first activity level ([second activity level]/[first activity level]) and its inverse ratio ([first activity level]/[second activity level]).

In the method in Embodiment 1, it can be determined that for example, when the difference between first activity level and second activity level ([first activity level]−[second activity level]) is smaller than a predetermined threshold value, the tumor cell to be evaluated is non-chemosensitive to anthracycline. When the difference is not smaller than the threshold value, the tumor cell can be judged to be chemosensitive to anthracycline.

When the ratio of second activity level to first activity level ([second activity level]/[first activity level]) is not lower than a predetermined threshold value, the tumor cell can be judged to be non-chemosensitive to anthracycline. When the ratio is lower than the threshold value, the tumor cell can be judged to be chemosensitive to anthracycline.

The threshold value can be established on the basis of a plurality of measurement data on degrees of change and chemosensitivities of tumor cells to anthracycline. Specifically, the threshold value can be established in the following manner. Whether tumor cells collected from a specific patient are chemosensitive to anthracycline is experimentally verified according to a conventional method. Using the same sample, the degree of change between first activity level and second activity level is calculated by the method in this embodiment. Samples from a plurality of patients are subjected to this procedure, whereby the value by which whether the cells are chemosensitive or not can be determined with the highest probability can be previously established as the threshold value. This threshold value is provided to the practitioner before practicing the method in this embodiment. When patient's tumor cells of unknown chemosensitivity to anthracycline are used as the sample, whether the cells are chemosensitive or not can be determined based on the threshold value, by the method in this embodiment without necessity for verification by the conventional method.

For example, if the concentration of anthracycline in the medium is 100 nM, and ([first activity level]−[second activity level]) is used as the degree of change, then the threshold value can be established between 70 and 370. The threshold value is established particularly preferably between 100 and 300. At this time, when ([first activity level]−[second activity level]) is lower than the threshold value, the tumor cell can be judged to be non-chemosensitive to anthracycline. On the other hand, when this difference is higher than the threshold value, the tumor cell can be judged to be chemosensitive.

If the concentration of anthracycline in the anticancer agent-containing medium is 100 nM, and ([second activity level]/[first activity level]) is used as the degree of change, then the threshold value can be established between 0.65 and 0.9. The threshold value is established particularly preferably between 0.7 and 0.8. At this time, when [second activity level]/[first activity level]) is higher than the threshold value, the tumor cell can be judged to be non-chemosensitive to anthracycline. On the other hand, when this ratio is lower than the threshold value, the tumor cell can be judged to be chemosensitive.

### Embodiment 2

The method in Embodiment 2 of the present invention is a method for determining . . . . (Claim)

In the first measurement step, the activity level and expression level of CDK1 contained in a first tumor cell (tumor cell not treated with anthracycline) are measured.

The activity level can be measured in the same manner as in Embodiment 1.

The expression level of CDK1 is the amount (unit corresponding to the number of molecules) of CDK1 contained in a cell lysate and can be measured by a method known in the art. For example, ELISA, western blotting, or the like may be used, or a method disclosed in U.S. Patent Application Publication No. 2004-214180 can be used in measurement. CDK1 can be captured with an anti-CDK1 antibody. Accordingly, the expression level of CDK1 contained in a cell lysate can be determined by measuring the amount of CDK1 captured with an anti-CDK1 antibody.

In the second measuring step, the activity level and expression level of CDK1 contained in a tumor cell treated with anthracycline are measured. The treatment of the tumor cell with anthracycline can be carried out in the same manner as in the second measuring step in the method in Embodiment 1. The activity level and expression level of CDK1 can be measured in the same manner as in the first measuring step in Embodiment 2.

Then, on the basis of the activity level and expression level of CDK1 obtained in the first measuring step and the activity level and expression level of CDK1 obtained in the second measuring step, the specific activity of CDK1 in the tumor cell not treated with anthracycline and the specific activity of CDK1 in the tumor cell treated with anthracycline are calculated respectively. The specific activity of CDK1 in the tumor cell not treated with anthracycline is referred to as first specific activity, and the specific activity of CDK1 in the tumor cell treated with anthracycline is referred to as second specific activity. As used herein, the specific activity is a value indicative of the ratio between the activity level and the expression level and can be calculated for example as the activity level/expression level ratio. The specific activity corresponds to the ratio of active CDK1 to CDK1 present in the cell.

On the basis of the specific activities obtained in the calculation step, the chemosensitivity of the tumor cell to anthracycline is determined.

In the method in Embodiment 2, the judgment step is preferably a step of judging the chemosensitivity of the tumor cell to anthracycline, based on the degree of change, by anthracycline, in the specific activity level of CDK1.

The degree of change in the method in Embodiment 2 is indicative of the degree of change between first specific activity and second specific activity. This degree of change includes, for example, the difference between first specific activity and second specific activity and the ratio between second specific activity and first specific activity. The difference between first specific activity and second specific activity includes ([first specific activity]−[second specific activity]) and ([second specific activity]−[first specific activity]). The ratio between first specific activity and second specific activity includes the ratio of second specific activity to first specific activity ([second specific activity]/[first specific activity]) and its inverse ratio ([first specific activity]/[second specific activity]).

When ([first specific activity]−[second specific activity]) is used as the degree of change, it can be judged that the tumor cell is non-chemosensitive to anthracycline when ([first specific activity]−[second specific activity]) is lower than a predetermined threshold value. When this difference is not lower than the threshold value, the tumor cell can be judged to be chemosensitive to anthracycline.

When ([second specific activity]/[first specific activity]) is used as the degree of change, it can be judged that the tumor cell is non-chemosensitive to anthracycline when ([second specific activity]/[first specific activity]) is not lower than a predetermined threshold value. On the other hand, the tumor cell can be judged to be chemosensitive to anthracycline when this ratio is lower than the threshold value.

In the judgment step, the chemosensitivity of the tumor cell to anthracycline is judged preferably by comparing the degree of change with a threshold value. The threshold value can be established in the same manner as for the threshold value established for the degree of change in CDK1 activity level in Embodiment 1.

For example, if the concentration of anthracycline in the anticancer agent-containing medium is 100 nM, and ([first specific activity]−[second specific activity]) is used as the degree of change, then the threshold value can be established between 4 and 21. The threshold value is established particularly preferably between 5 and 15. At this time, when ([first specific activity]−[second specific activity]) is lower than the threshold value, the tumor cell can be judged to be non-chemosensitive to anthracycline. On the other hand, when this difference is not lower than the threshold value, the tumor cell can be judged to be chemosensitive to anthracycline.

If the concentration of anthracycline in the anticancer agent-containing medium is 100 nM, and ([second specific activity]/[first specific activity]) is used as the degree of change, then the threshold value can be established between 0.6 and 0.9. The threshold value is established particularly preferably between 0.7 and 0.8. At this time, when [second specific activity]/[first specific activity]) is higher than the threshold value, the tumor cell can be judged to be non-chemosensitive to anthracycline. On the other hand, when this ratio is not higher than the threshold value, the tumor cell can be judged to be chemosensitive to anthracycline.

### 2. Apparatus for Judging Chemosensitivity to Anthracycline

Hereinafter, the apparatus for determining chemosensitivity to anthracycline is described with reference to the drawings.

FIG. 10 is a block diagram showing a schematic configuration of the apparatus in this embodiment. The apparatus 1 is an apparatus for determining the chemosensitivity of tumor cells to anthracycline and is composed of an information processing device 10 and a measurement device 20 as shown in FIG. 10. The information processing device 10 is a device that analyzes measurement data outputted by the measurement device 20 to judge the chemosensitivity of tumor cells to anthracycline, and outputs the judgment result. The measurement device 20 is a device for measurement of the activity level and expression level of CDK1 in tumor cells and outputs the measurement data to the information processing device 10.

The information processing device 10 has a personal computer (PC) and includes an information processing unit 101, a keyboard 103 and a display unit 105. The information processing unit 101 includes CPU 101a, ROM 101b, RAM 101c, a hard disk 101d, an I/O interface 101e, a communication interface 101f, an image output interface 101g, and bus 101h, and the respective parts are connected to one another via bus 101h to mutually transmit and receive data.

The CPU 101a can execute computer programs stored in the ROM 101b and computer programs loaded in the RAM 101c. The ROM 101b stores computer programs executed by the CPU 101a and data used for executing these computer programs by the CPU 101a. The RAM 101c is used to read out computer programs stored in the ROM 101b and hard disk 101d. The RAM 101c is also used as a work area for the CPU 101a when executing these computer programs.

Various computer programs to be executed by the CPU 101a, such as an operating system (OS) and application programs, and data used for executing these computer programs, are installed on the hard disk 101d.

An application program 101i installed on the hard disk 101d contains an application program for realizing the method of judging the chemosensitivity of tumor cells to anthracycline. The hard disk 101d stores a threshold value as data used in execution of the application program realizing the method of judging the chemosensitivity of tumor cells to anthracycline.

The I/O interface 101e has a keyboard 103 and a mouse (not shown) connected thereto. A communication interface 101f has the measurement device 20 connected thereto, and via the communication interface 101f, data can be transmitted and received between the information processing unit 101 and the measurement device 2.

The image output interface 101g is connected to the display unit 105 and outputs an image signal corresponding to image data given by the CPU 101a to the display unit 105. The display unit 105 displays an (on-screen) image according to the input image signal.

The measurement device 20 connected via the communication interface 101f to the information processing device 10 is a device for measuring the activity level and expression level of CDK1 from a biological tissue. As the measurement device, a device described in U.S. Patent Application Publication No. 2007-0077658 can be used.

The apparatus 1 is constituted in this embodiment so as to judge the chemosensitivity of tumor cells to anthracycline, on the basis of measurement data obtained by the measurement device 20, but may, without limitation to such constitution, be constituted such that measurement data on the previously measured activity level and expression level of CDK1 are inputted via the keyboard 103.

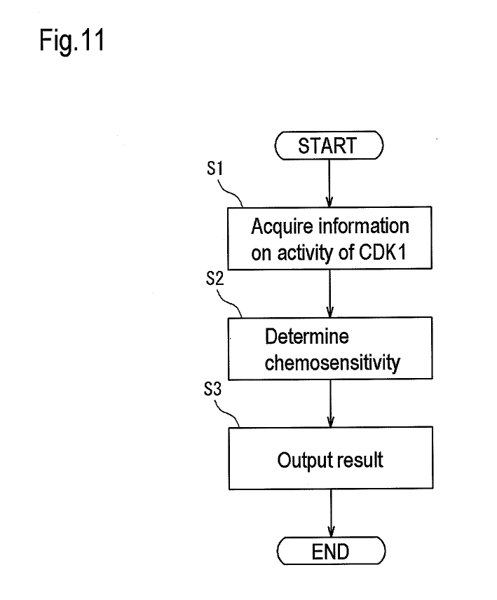

The apparatus 1 in this embodiment includes a program for judging the sensitivity of tumor cells to anthracycline. FIG. 11 is a flowchart showing processing executed by this program.

First, in step S1, the measurement device 20 acquires information on the activity of CDK1 in a tumor cell not treated with anthracycline and information on the activity of CDK1 in a tumor cell treated with anthracycline. Specifically, the measurement device 20 measures the activity level and expression level of CDK1 as information on the activity of CDK1, thereby acquiring the measurement data. Hereinafter, the information on the activity of CDK1 in the tumor cell not treated with anthracycline is referred to first information, and the information on the activity of CDK1 in the tumor cell treated with anthracycline is referred to second information. The first information and second information acquired by the measurement device 20 are transmitted via the communication interface 101f to the information processing unit 101. The information on activity includes, for example, activity level and specific activity.

In step S2, CPU 101a in the information processing unit 101 executes an application program, installed on the hard disk 101d, for realizing the method of determining the chemosensitivity of the tumor cell to anthracycline, thereby determining the chemosensitivity of the tumor cell to anthracycline. Specifically, the degree of change in CDK1 activity between the tumor cell not treated with anthracycline and the tumor cell treated with anthracycline is calculated from the first information and second information. The calculated degree of change is compared with a threshold value stored in the hard disk 101d. Then, on the basis of the comparison result, the chemosensitivity of the tumor cell to anthracycline is judged.

Examples of the degree of change herein include:
(1) the difference between the activity level of CDK1 based on first information and the activity level of CDK1 based on second information;
(2) the ratio between the activity level of CDK1 based on first information and the activity level of CDK1 based on second information;
(3) the difference between the specific activity of CDK1 based on first information and the specific activity of CDK1 based on second information; and
(4) the ratio between the specific activity of CDK1 based on first information and the specific activity of CDK1 based on second information.

The activity level and specific activity of CDK1 can be obtained from the measurement data on the activity level and expression level of CDK1 measured in the measurement device 20. The threshold value can be appropriately established depending on the degree of change used in judgment.

Then, in step S3, the judgment result is transmitted from the image output interface 101g in the information processing unit 101 to the display unit 105, thereby displaying the judgment result on the display unit 105.

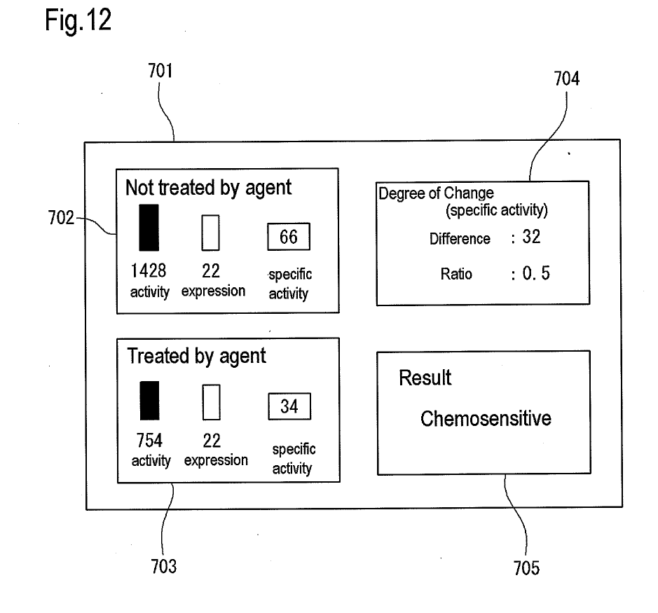

FIG. 12 shows a screen (window) displayed in the display unit 105 in the apparatus 1 shown in FIG. 10. The window 701 in FIG. 12 displays information 702 on the activity of CDK1 in the tumor cell not treated with an anticancer agent (anthracycline anticancer agent), information 703 on the activity of CDK1 in the tumor cell treated with an anticancer agent (anthracycline anticancer agent), information 704 on the degree of change, and information 705 on the judgment result.

The information 702 indicates the activity level, expression level and specific activity of CDK1 in the tumor cell not treated with anthracycline. The information 703 indicates the activity level, expression level and specific activity of CDK1 in the tumor cell treated with anthracycline. The information 704 indicates the degree of change, that is, the difference or ratio between the specific activities. The information 704 indicates the difference between the specific activities, that is, ([first specific activity]−[second specific activity,]). As the ratio between the specific activities, ([second specific activity]/[first specific activity]) is indicated. The information 705 indicates the result of judgment of the chemosensitivity of the tumor cell to anthracycline, which is based on the comparison result.

On the basis of information 702, information 703, information 704 and information 705 displayed on screen 701, the user of the apparatus can select an anticancer agent to be used in the patient.

### Experiments

### REFERENCE EXAMPLE 1

Human breast cancer cell lines, that is, HS 578T cells (ATCC HTB-126R), MDA-MB-435s cells (ATCC HTB-129), BT20 cells (ATCC HTB-19), CAMA-1 cells (ATCC HTB-21), T47D cells (ATCC HTB-133), MDA-MB-361 cells (ATCC HTB-27), UACC893 cells (ATCC CRL-1902) and BT474 cells (ATCC HTB-20) were purchased from American Type Culture Collection (ATCC) and cultured under culture conditions recommended by ATCC, as follow.

HS 578T cells were cultured in 5 vol % CO₂ at 37° C., in a Dulbecco's modified Eagle medium containing 4 mM of L-glutamine, 10% by mass of fetal bovine serum and 0.01 mg/L of bovine insulin, with glucose at a final concentration of 4.5 g/L and sodium bicarbonate at a final concentration of 1.5/L.

MDA-MD-435S cells were cultured in an air atmosphere at 37° C., in a Liebovitz L15 medium containing 2 mM of L-glutamine, 10% by mass of fetal bovine serum and 0.01 mg/L of insulin.

BT-20 cells were cultured in 5 vol % CO₂ at 37° C., in an Eagle minimum essential medium containing Earle's BSS, 2 mM of L-glutamine, 1.0 mM of sodium pyruvate, 0.1 mM of nonessential amino acids, 1.5 g/L of sodium bicarbonate and 10% by mass of fetal bovine serum.

CAMA-1 cells were cultured in 5 vol % CO₂ at 37° C., in an Eagle minimum essential medium containing Earle's BSS, 2 mM of L-glutamine, 1.0 mM of sodium pyruvate, 0.1 mM of nonessential amino acids, 1.5 g/L of sodium bicarbonate and 10% by mass of fetal bovine serum.

T-47D cells were cultured in 5 vol % CO₂ at 37° C., in an RPMI 1640 medium containing 2 mM of L-glutamine, 1.0 mM of HEPES, 1.0 mM of sodium pyruvate, 4.5 g/L of glucose, 1.5 g/L of sodium bicarbonate, 10% by mass of fetal bovine serum and 0.2 unit/mL of bovine insulin.

MDA-MB-361 cells were cultured in an air atmosphere at 37° C., in a Liebovitz L15 medium containing 20% by mass of fetal bovine serum.

UACC-893 cells were cultured in an air atmosphere at 37° C., in a Liebovitz L15 medium containing 2 mM L-glutamine, 10% by mass of fetal bovine serum and 0.01 mg/L of insulin.

BT-474 cells were cultured in 5 vol % CO₂ at 37° C., in a Dulbecco's modified medium containing 10% by mass of fetal bovine serum.

### REFERENCE EXAMPLE 2

Each of the breast cancer cell lines, that is, HS 578T cells, MDA-MB-435S cells, BT20 cells, CAMA-1 cells, T47D cells, MDA-MB-361 cells, UACC893 cells and BT474 cells, was diluted stepwise to produce 7 serial cell dilutions. Each of the serial cell dilutions was inoculated into its corresponding medium and cultured for 24 hours under the culture conditions shown in Reference Example 1.

The number of living cells in each of the cultures thus obtained was determined by an MTT method of using 3-(4,5-dimethylthiazol-2-yl)-2,5-diphenyl-2H-tetrazolium bromide. The number of living cells to be inoculated was established for each type of cell so as to enable the influence of the drug to be evaluated in the measurement range where the primary correlation between the number of cells in the serial dilution to be inoculated and the determined number of the living cells was kept high. The cells were inoculated into the medium such that the established number of the living cells was reached in the medium upon inoculation, and then the cells were cultured in an incubator for 24 hours under the culture conditions shown in Reference Example 1.

1 μM of doxorubicin was diluted stepwise to produce 8 serial dilutions, and each of the resulting serial dilutions of doxorubicin was added to the medium of each type of cell, and the cells were cultured for 3 days under the culture conditions shown in Reference Example 1. As the control, each type of cell was cultured in the same manner as above except that the serial dilution of doxorubicin was replaced by the same volume of the medium.

Thereafter, the number of living cells in each of the cultures thus obtained was measured by the MTT method of using 3-(4,5-dimethylthiazol-2-yl)-2,5-diphenyl-2H-tetrazolium bromide. This measurement was conducted 4 times.

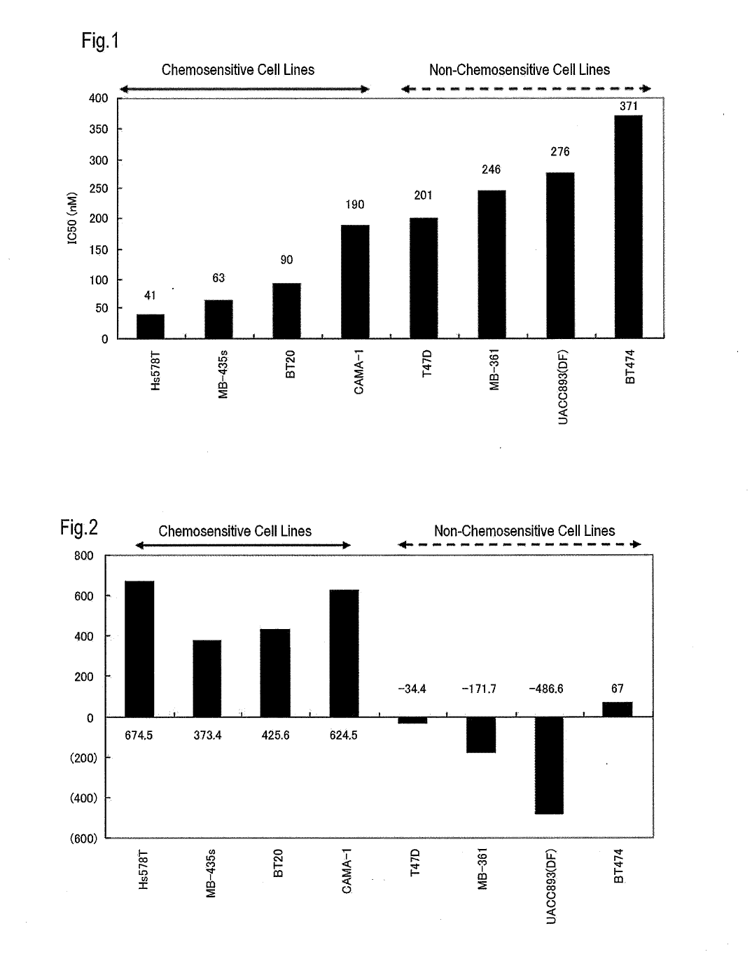

On the basis of the determined number of living cells, the concentration of doxorubicin (IC50) at which the ratio of the number of living cells in the culture not treated with doxorubicin to the number of living cells in the culture treated with doxorubicin became 50% was determined. The results are shown in FIG. 1.

It is known that the effective blood concentration of doxorubicin used generally in chemotherapy is about 200 nM. It was thus assumed that a breast cancer cell line that IC50 being not lower than 200 nM (i.e. the blood concentration of doxorubicin) was a non-chemosensitive strain, and a breast cancer cell line that IC50 being lower than 200 nM was a chemosensitive strain.

### EXAMPLE 1

### (1) Doxorubicin Treatment

Each of the breast cancer cell lines cultured under the culture conditions shown in Reference Example 1 was inoculated into a medium in a T-225 flask at a density of 1×10⁷ cells per flask and then cultured for 24hours. Thereafter, the medium for the resulting culture was exchanged with 40 mL of medium containing 100 nM of doxorubicin, and the cells were cultured for additional 24 hours. Thereafter, the medium was removed from the flask, and 15 mL of phosphate buffered physiological saline was introduced into the flask and used to wash the cells in the flask. Thereafter, 3 mL of trypsin-EDTA solution (0.25 mass % of trypsin and 1 mM of EDTA) was added to the cells in the flask followed by incubation at 37° C. for 5 minutes to detach the cells from the flask. 10 mL or more of medium was added to this flask to terminate the trypsin reaction, and the cells were recovered. The recovered cells were centrifuged at 190×g for 5 minutes, and the supernatant was removed. The obtained cells were suspended in a suitable amount of phosphate buffered physiological saline. The resulting cell suspension was centrifuged at 190×g for 5 minutes, and the supernatant was removed. The activity level and expression level of CDK1 in the obtained cells were determined in the following manner. The obtained cells were frozen in liquid nitrogen and stored at −80° C. prior to measurement of the activity level and expression level of CDK1.

The obtained cells were suspended in an amount of about 150 mg cells/mL in buffer A (composition: 0.1 w/v % of Nonidet P-40 (manufactured by Calbiochem), 50 mM of Tris-HCl buffer (pH 7.4), 5 mM of EDTA, 50 mM of sodium fluoride, 1 mM of sodium o-vanadate, and 100 μl/ml of proteinase inhibitor cocktail (Sigma)) to produce a cell suspension. Thereafter, the cells in the cell suspension were disrupted to prepare a cell lysate. Then, the cell lysate was centrifuged at 15000 rpm for 5 minutes at 4° C. to give a supernatant. The resulting supernatant was used as a doxorubicin-treated sample.

A doxorubicin-untreated sample was obtained in the same manner as above except that a doxorubicin-free medium was used in place of the medium containing 100 nM of doxorubicin.

The doxorubicin-treated sample and the doxorubicin-untreated sample were used as CDK1 measurement samples.

### (3) Measurement of the Activity Level of CDK1

The activity level of CDK1 was measured as follow: 500 μL of buffer A was added to a 5-mL microtube (manufactured by Eppendorf) and then the CDK measurement sample was added such that the total protein mass reached 100 μg per microtube.

Sepharose beads (Bio-Rad) coated with 2 μg of anti-CDK1 antibody and 20 μg of Protein A were added to the resulting mixture and left at 4° C. for 1 hour thereby reacting CDK1 with the anti-CDK1 antibody.

After the reaction, the beads were washed 3 times with a beads washing buffer (composition: 0.1 w/v % of Nonidet P-40 and 50 mM of tris-HCl, pH 7.0) and then re-suspended in 15 μL of lysing buffer A to give a sample containing Sepharose beads to which CDK1 had been bound via the anti-CDK1 antibody.

A substrate solution for CDK1 (composition: 10 μg of histone H1, 5 mM of ATP-γS (manufactured by Sigma), 20 mM of Tris-HCl (pH 7.4), 0.1 w/v % Triton X-100) was added to this mixture such that the total amount of the resulting mixture reached 50 μL. The resulting mixture was subjected to kinase reaction under shaking at 37° C. for 10 minutes thereby introducing a monothiophosphate group into histone H1.

After the kinase reaction, the mixture was centrifuged at 2,000 rpm for 20 seconds to precipitate the beads, and 18 μL of supernatant was recovered. 15 μl of a binding buffer (composition: 150 mM of Tris-HCl (pH 9.2) and 5 mM of EDTA) and 10 mM of iodoacetylbiotin solution (composition: 10 mM of iodoacetylbiotin, 100 mM of Tris-HCl (pH 7.5) and 1 mM of EDTA) were added to the resulting supernatant. The resulting mixture was left for 90 minutes at room temperature in a dark place, thereby binding iodoacetylbiotin to a sulfur atom of the substrate (monothiophosphorylated substrate) having a monothiophosphate group introduced into it.

A sample containing 0.4 μg of the monothiophosphorylated substrate to which iodoacetylbiotin had been bound was blotted onto a PVDF membrane by means of a slot blotter. After blotting, the PVDF membrane was blocked with a solution containing 1 w/v % of BSA, and then streptavidin-FITC (manufactured by Vector Laboratories Inc.) was added thereto followed by incubation at 3720 C. for 1 hour. Thereafter, the PVDF membrane was washed 3 times with 50 mM of washing solution B. After washing, the PVDF membrane was subjected to fluorescence analysis with a fluorescence image analyzer Molecular Imager FX (Bio-Rad Laboratories, Inc.). The activity level of CDK1 was determined based on a calibration curve. The calibration curve was prepared by blotting a solution containing a protein (biotin-labeled immunoglobulin) at 2 types of different concentrations, onto the PVDF membrane, to label the membrane with FITC in the same manner as described above, and measuring the fluorescence intensity of the protein with the fluorescence image analyzer. 1 U (unit) activity of the measured CDK1 denotes a value indicating fluorescence intensity equal to the fluorescence of 1 ng of the protein.

The difference between the activity level of CDK1 in the breast cancer cell not treated with doxorubicin and the activity level of CDK1 in the breast cancer cell treated with doxorubicin was calculated using the following formula: (the activity level of CDK1 in the breast cancer cell not treated with doxorubicin)−(the activity level of CDK1 in the breast cancer cell treated with doxorubicin). The results are shown in FIG. 2.

The results shown in FIG. 2 indicate that in breast cancer cell lines that were estimated by IC50 to be chemosensitive strains, that is, HS 578T cells, MDA-MB-435S cells, BT20 cells and CAMA-1 cells, there is a great difference between the activity level of CDK1 in the breast cancer cells not treated with doxorubicin and the activity level of CDK1 in the breast cancer cells treated with doxorubicin. On the other hand, it can be seen that in breast cancer cell lines that were estimated by IC50 to be non-chemosensitive strains, that is, T47D cells, MDA-MB-361 cells, UACC893 cells and BT474 cells, there is a little difference, or a minus difference, between the activity level of CDK1 in the breast cancer cells not treated with doxorubicin and the activity level of CDK1 in the breast cancer cells treated with doxorubicin. From the foregoing, it is suggested that the chemosensitivity of the tumor cell to anthracycline can be judged based on the difference between the activity level of CDK1 in the tumor cell not treated with anthracycline represented by doxorubicin and the activity level of CDK1 in the tumor cell treated with anthracycline.

### EXAMPLE 2

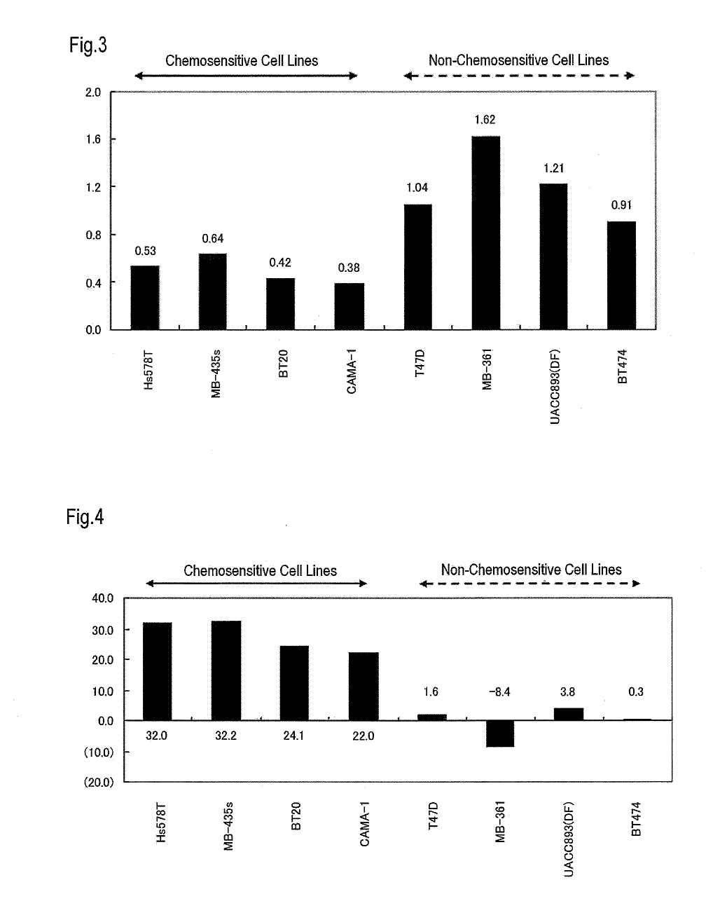

The activity level of CDK1 in the breast cancer cell not treated with doxorubicin and the activity level of CDK1 in the breast cancer cell treated with doxorubicin were measured in the same manner as in Example 1. Then, the ratio between the activity level of CDK1 in the breast cancer cell not treated with doxorubicin and the activity level of CDK1 in the breast cancer cell treated with doxorubicin was calculated from the formula: (the activity level of CDK1 in the breast cancer cell treated with doxorubicin)/(activity level of CDK1 in the breast cancer cell not treated with doxorubicin). The results are shown in FIG. 3.

The results shown in FIG. 3 indicate that in beast tumor cell lines judged by IC50 to be chemosensitive strains, that is, HS 578T cells, MDA-MB-435S cells, BT20 cells and CAMA-1 cells, the ratio between the activity level of CDK1 in the breast cancer cells not treated with doxorubicin and the activity level of CDK1 in the breast cancer cells treated with doxorubicin is low. On the other hand, it can be seen that in breast cancer cell lines judged by IC50 to be non-chemosensitive strains, that is, T47D cells, MDA-MB-361 cells, UACC893 cells and BT474 cells, the ratio between the activity level of CDK1 in the breast cancer cells not treated with doxorubicin and the activity level of CDK1 in the breast cancer cells treated with doxorubicin is high. From the foregoing, it is suggested that the chemosensitivity of the tumor cell to anthracycline can be judged based on the ratio between the activity level of CDK1 in the tumor cell not treated with anthracycline represented by doxorubicin and the activity level of CDK1 in the tumor cell treated with anthracycline.

### EXAMPLE 3

### (1) Measurement of the Activity Level of CDK1

The activity level of CDK1 in the tumor cell not treated with doxorubicin and the activity level of CDK1 in the tumor cell treated with doxorubicin were measured in the same manner as in Example 1.

### (2) Measurement of the Expression Level of CDK1

The expression level of CDK1 was measured as follow: 50 μl of each of CDK1 measurement samples was put into each well of a blotter in which a PVDF membrane (Millipore Corporation) had been set. Then, the sample was suctioned at a negative pressure of approximately 250 mmHg (33.3 Pa) from the bottom of the well, that is, the rear surface of the membrane for about 30 seconds so that the protein in the CDK1 measurement sample was adsorbed onto the PVDF membrane.

100 μl of a washing solution B (composition: 25 mM of Tris-HCl (pH 7.4) and 150 mM of NaCl) was put into each well, and the washing solution B was suctioned at a negative pressure 500 mmHg (66.6 Pa) from the rear surface of the membrane for 15 seconds so that the membrane was washed.

Then, 40 μl of a blocking reagent B (composition: 4% BSA, 25 mM of Tris-HCl (pH 7.4) and 150 mM of NaCl) was put into each well and the membrane was left in a stationary state for 15 minutes. Thereafter, the blocking reagent B was suctioned at a negative pressure of 500 mmHg (66.6 Pa) from the rear surface of the membrane for 15 seconds so that the membrane was blocked.

Then, 40 μL of a rabbit anti-CDK1 antibody solution (primary antibody) was added to each well, and then the membrane was left at room temperature for about 30 minutes, whereby CDK1 on the membrane was reacted with the primary antibody. Thereafter, the solution was suctioned at a negative pressure of 500 mmHg from the bottom of the well for about 15 seconds. Then, 100 μL of washing solution B was placed on the well and the membrane was washed by suction at a negative pressure of 500 mmHg (66.6 Pa) for about 15 seconds.

Then, 40 μl of a biotinylated anti-rabbit IgG antibody (secondary antibody) solution was put into each well and left in a stationary state at room temperature for about 30 minutes so that the primary and secondary antibodies in the membrane were reacted with each other. Then, the solution was suctioned at a negative pressure of 500 mmHg (66.6 Pa) from the bottom of the well for about 15 seconds.

Thereafter, 100 μl of the washing solution B was put into each well and was suctioned at a negative pressure of 500 mmHg (66.6 Pa) for 15 seconds so that the membrane was washed. Then, 50 μl of a label solution containing FITC-labeled streptavidin was put into each well and left at room temperature for about 30 minutes to label the secondary antibody on the membrane with FITC, and then was suctioned at a negative pressure of 500 mmHg (66.6 Pa) from the bottom of the membrane for 15 seconds.

Thereafter, 100 μl of the washing solution B was put into each well, and was suctioned at a negative pressure of 500 mmHg (66.6 Pa) for 15 seconds so that the membrane was washed. The washing of the membrane with the washing solution B was conducted repeatedly 5 times.

After washing, the membrane was removed from the blotter and rinsed with 20 volt % of methanol for about 5 minutes. Thereafter, the membrane was dried at room temperature for about 20 minutes, and then the fluorescence intensity based on the protein adsorbed onto the membrane was measured with a fluorescence image analyzer, Molecular Image FX (Bio-Rad Laboratories, Inc.). The activity level of CDK1 was determined based on a calibration curve. The calibration curve was prepared by adding 50 μl of each of solutions containing recombinant CDK1 at 5 types of different concentrations in the washing solution B containing 0.005% Nonidet P-40 and 50 μg/ml of BSA, to each well treated in the same manner as described above, then labeling it with FITC labeling in the same experimental procedures as described above, and measuring the fluorescence intensity so that the relationship between the fluorescence intensity and the CDK1 expression level of CDK1 was expressed as a calibration curve.

### (3) Calculation of Specific Activity

From the measured activity level and expression level of CDK1, CDK1 specific activity (mU/ng) was calculated from the equation: CDK1 specific activity=CDK1 activity level/CDK1 expression level.

The difference between the specific activity of CDK1 in the breast cancer cell not treated with doxorubicin and the specific activity of CDK1 in the breast cancer cell treated with doxorubicin was calculated from the formula: (specific activity of CDK1 in the breast cancer cell not treated with doxorubicin)−(specific activity of CDK1 in the breast cancer cell treated with doxorubicin). The results are shown in FIG. 4.

The results shown in FIG. 4 indicate that in the beast tumor cell lines judged by IC50 to be chemosensitive strains, that is, HS 578T cells, MDA-MB-435S cells, BT20 cells and CAMA-1 cells, the difference between the specific activity of CDK1 in the breast cancer cells not treated with doxorubicin and the specific activity of CDK1 in the breast cancer cells treated with doxorubicin is high. On the other hand, it can be seen that in the breast cancer cell lines judged by IC50 to be non-chemosensitive strains, that is, T47D cells, MDA-MB-361 cells, UACC893 cells and BT474 cells, there is a little difference, or a minus difference, between the specific activity of CDK1 in the breast cancer cells not treated with doxorubicin and the specific activity of CDK1 in the breast cancer cells treated with doxorubicin. From the foregoing, it is suggested that the chemosensitivity of the tumor cell to anthracycline can be judged based on the difference between the specific activity of CDK1 in the tumor cell not treated with anthracycline represented by doxorubicin and the specific activity level of CDK1 in the tumor cell treated with anthracycline.

### EXAMPLE 4

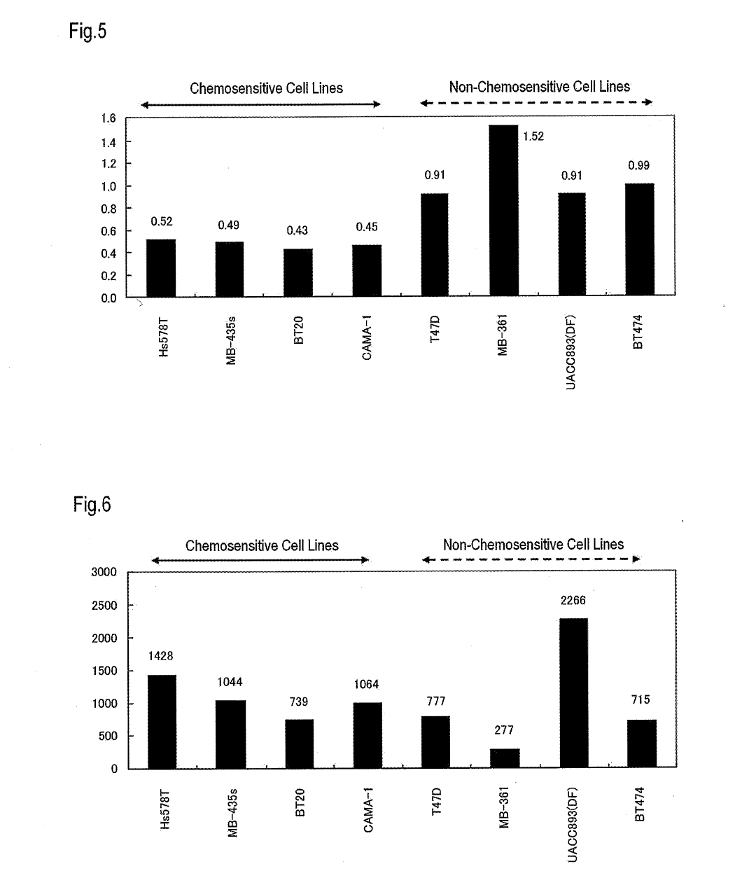

The activity level and expression level of CDK1 in the breast cancer cell not treated with doxorubicin as well as the activity level and expression level of CDK1 in the breast cancer cell treated with doxorubicin were measured in the same manner as in Example 3. Then, the ratio between the specific activity of CDK1 in the breast cancer cell not treated with doxorubicin and the specific activity of CDK1 in the breast cancer cell treated with doxorubicin was calculated from the formula: (specific activity of CDK1 in the breast cancer cell treated with doxorubicin)/(specific activity of CDK1 in the breast cancer cell not treated with doxorubicin). The results are shown in FIG. 5.

The results shown in FIG. 5 indicate that in the beast tumor cell lines judged by IC50 to be chemosensitive strains, that is, HS578T cells, MDA-MB-435S cells, BT20 cells and CAMA-1 cells, the ratio between the specific activity of CDK1 in the breast cancer cells not treated with doxorubicin and the specific activity of CDK1 in the breast cancer cells treated with doxorubicin is low. On the other hand, it can be seen that in the breast cancer cell lines judged by IC50 to be non-chemosensitive strains, that is, T47D cells, MDA-MB-361 cells, UACC893 cells and BT474 cells, the ratio between the specific activity of CDK1 in the breast cancer cells not treated with doxorubicin and the specific activity of CDK1 in the breast cancer cells treated with doxorubicin is high. From the foregoing, it is suggested that the chemosensitivity of the tumor cell to anthracycline can be judged based on the ratio between the specific activity of CDK1 in the tumor cell not treated with anthracycline represented by doxorubicin and the specific activity of CDK1 in the tumor cell treated with anthracycline.

### COMPARATIVE EXAMPLE 1

The activity level of CDK1 in the breast cancer cell not treated with doxorubicin was measured by the same operation as in Example 1. The results are shown in FIG. 6.

From the results shown in FIG. 6, it can be seen that based on only the activity level of CDK1 in the breast cancer cell not treated with doxorubicin, it not possible to determine whether the cell is a chemosensitive strain or a non-chemosensitive strain.

### COMPARATIVE EXAMPLE 2

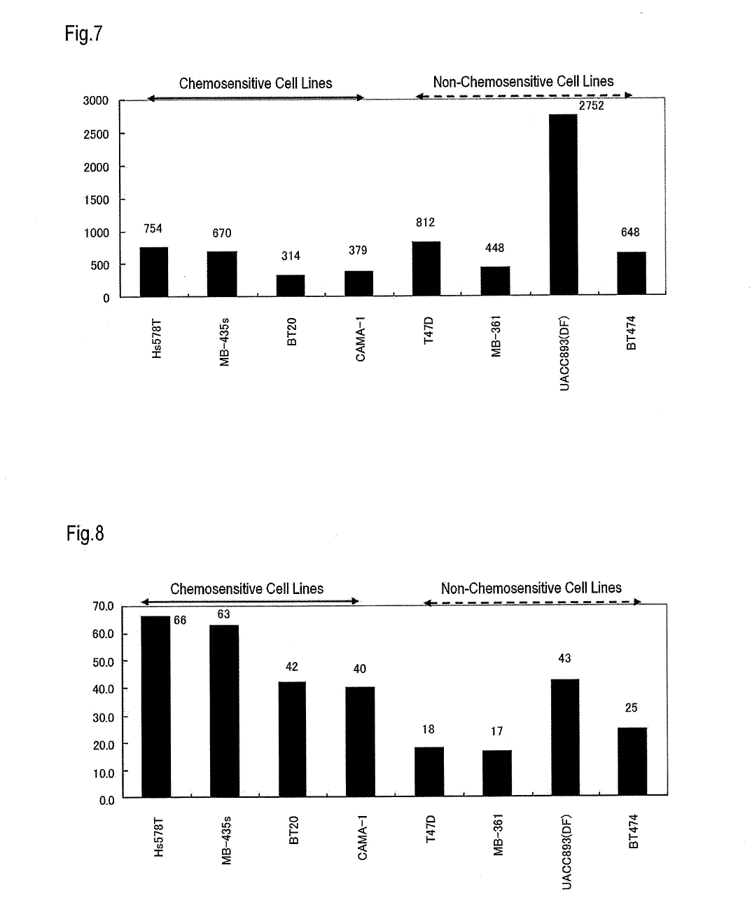

The activity level of CDK1 in the breast cancer cell treated with doxorubicin was measured by the same operation as in Example 1. The results are shown in FIG. 7.

From the results shown in FIG. 7, it can be seen that based on only the activity level of CDK1 in the breast cancer cells treated with doxorubicin, it is not possible to determine whether the cell is a chemosensitive strain or a non-chemosensitive strain.

### COMPARATIVE EXAMPLE 3

The activity level and expression level of CDK1 in the breast cancer cell not treated with doxorubicin were measured by the same operation as in Example 3, to determine the specific activity. The results are shown in FIG. 8.

From the results shown in FIG. 8, it can be seen that based on only the specific activity of CDK1 in the breast cancer cell not treated with doxorubicin, it is not possible to clearly determine whether the cell is a chemosensitive strain or a non-chemosensitive strain.

### COMPARATIVE EXAMPLE 4

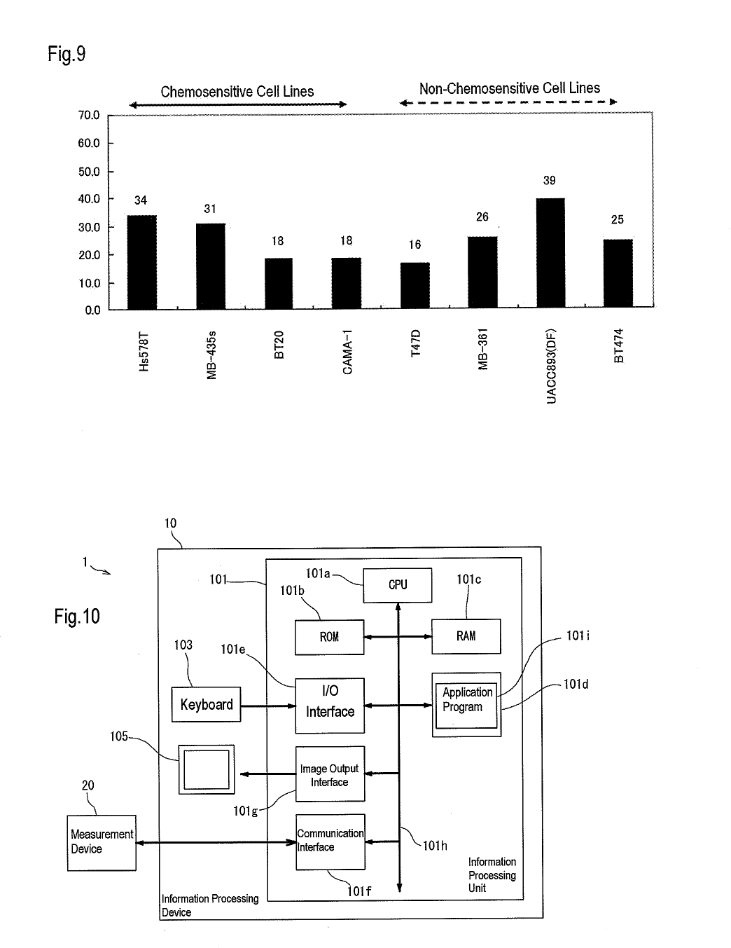

The activity level and expression level of CDK1 in the breast cancer cell treated with doxorubicin were measured by the same operation as in Example 3, to determine the specific activity. The results are shown in FIG. 9.

From the results shown in FIG. 9, it can be seen that based on only the specific activity of CDK1 in the breast cancer cell treated with doxorubicin, it is not possible to clearly determine whether the cell is a chemosensitive strain or a non-chemosensitive strain.

What is claimed is:

1. A method for determining chemosensitivity of a malignant tumor cell to anthracycline, comprising:
preparing first tumor cell and second tumor cell both of which are derived from a malignant tumor obtained from a subject, wherein the first tumor cell is not treated with anthracycline and the second tumor cell is treated with anthracycline;
measuring first activity level of CDK1 in the first tumor cell;
measuring second activity level of CDK1 in the second tumor cell; and
determining chemosensitivity of the malignant tumor cell based on the first and second activity levels.

2. The method according to claim 1, wherein the malignant tumor is breast cancer.

3. The method according to claim 1, wherein the anthracycline is daunorubicin, doxorubicin, pirarubicin, aclarubicin, epirubicin, oxaunomycin, or idarubicin

4. The method according to claim 1, wherein the determining step is performed by determining the chemosensitivity based on degree of change between the first activity level and second activity level.

5. The method according to claim 4, wherein the determining step is performed by comparing the degree of change to a predetermined threshold value, and determining the chemosensitivity based on result of the comparing.

6. The method according to claim 4, wherein the degree of change is difference or ratio between the first activity level and second activity level.

7. The method according to claim 1, wherein the determining step is performed by determining that the malignant tumor cell is chemosensitive when the second activity level is smaller than the first activity level by more than predetermined threshold value.

8. The method according to claim 1, wherein the determining step is performed by determining that the malignant tumor cell is chemosensitive when ratio of the second activity level to the first activity level is smaller than the predetermined threshold value.

9. A method for determining chemosensitivity of a malignant tumor cell to anthracycline, comprising:
preparing first tumor cell and second tumor cell both of which are derived from a malignant tumor obtained from same subject, wherein the first tumor cell is not treated with anthracycline and the second tumor cell is treated with anthracycline;
measuring first activity level and first expression level of CDK1 in the first tumor cell;
measuring second activity level and first expression level of CDK1 in the second tumor cell;
calclulating first specific activity and second specific activity, wherein the first specific activity is calculated from the first activity level and first expression level, and the second specific activity is calculated form the second activity level and first expression level;
determining chemosensitivity of the malignant tumor cell based on the first and second specific activities.

10. The method according to claim 9, wherein the determining step is performed by determining the chemosensitivity based on degree of change between the first specific activity and second specific activity.

11. The method according to claim 10, wherein the determining step is performed by comparing the degree of change to a predetermined threshold value, and determining the chemosensitivity based on a result of the comparing.

12. The method according to claim 10, wherein the degree of change is difference or ratio between the first specific activity and second specific activity.

13. The method according to claim 9, wherein the determining step is performed by determining that the malignant tumor cell is chemosensitive when the second specific activity is smaller than the first specific activity by more than predetermined threshold value.

14. The method according to claim 9, wherein the determining step is performed by determining that the malignant tumor cell is chemosensitive when ratio of the second specific activity to the first specific activity is smaller than the predetermined threshold value.

15. An apparatus for determining chemosensitivity of a malignant tumor cell to anthracycline, comprising:
an information obtaining part for obtaining first information and second information, wherein the first information is information related to activity level of CDK1 in first tumor cell which is not treated with anthracycline, and the second information is information related to activity level of CDK1 in second tumor cell which is treated with anthracycline, and wherein the first tumor cell and second tumor cell are derived from a malignant tumor obtained from same subject,
a determining part for determining chemosensitivity of the malignant tumor cell based on the first and second information, and
a display for outputting result of the determining.

16. The apparatus according to claim 15, wherein the determining part calculates degree of change between the first activity level and second activity level.

17. The apparatus according to claim 16, further comprising a memory for memorizing a predetermined threshold value, wherein the determining part compares the calculated degree of change to the predetermined threshold value, and determines the chemosensitivity based on result of the comparing.

18. The apparatus according to claim 16, wherein the degree of change is difference or ratio between the first activity level and second activity level.

19. The apparatus according to claim 15, wherein the determining part determines that the malignant tumor cell is chemosensitive when the second activity level is smaller than the first activity level by more than predetermined threshold value.

20. The apparatus according to claim 15, wherein the determining part determines that the malignant tumor cell is chemosensitive when ratio of the second activity level y to the first activity level is smaller than the predetermined threshold.
